(19) United States
(12) Patent Application Publication
(10) Pub. No.: US 2022/0072177 A1
(43) Pub. Date: Mar. 10, 2022

(54) HYGIENE SYSTEM AND METHOD FOR OPERATING A HYGIENE SYSTEM

(71) Applicant: Ideal Standard International NV, Zaventem (BE)

(72) Inventors: Bert Depiere, Haacht (BE); Ralf Donell, Morbach (DE); Rainer Mettler, Altrich (DE)

(21) Appl. No.: 17/467,153

(22) Filed: Sep. 3, 2021

(30) Foreign Application Priority Data
Sep. 8, 2020 (EP) 20195136.5

(51) Int. Cl. A61L 2/28; A47K 5/12; A61L 2/24; A61L 2/00; G08B 21/24; G08B 5/22

(52) U.S. Cl. CPC A61L 2/28; A47K 5/1217; A61L 2/24; A61L 2/0088; A61L 2202/17; G08B 5/22; A61L 2202/15; A61L 2202/14; G08B 21/245

(57) ABSTRACT

The present invention relates to a hygiene system comprising
at least one sanitary fitting (1), wherein the sanitary fitting has at least one fluid outlet (2.1, 2.2) for dispensing at least one fluid,
at least one camera (3) for recording the hands (6) of a user,
at least one optical display (4) and
a control (5), wherein the control is connected to the camera (3) and the optical display (4).

### RELATED APPLICATION

This application claims priority to European Patent Application No. 20195136.5, filed on Sep. 8, 2020. The entirety of such application is incorporated herein by reference.

### DESCRIPTION

The present invention relates to a hygiene system, comprising a sanitary fitting, wherein the sanitary fitting has at least one fluid outlet for dispensing a fluid (such as water, soap and/or disinfectant), a camera for recording the hands of a user, an optical display and a control, wherein the control is in data connection with the camera and with the display. The invention also relates to a method for operating a hygiene system, which includes the procedural steps of recording the hands of a user during a cleaning procedure and the processor-supported evaluation of the recording.

For hygienic cleaning of the hands and sufficiently reliable removal of viruses and bacteria from the hands, it is necessary for all areas of the hands (e.g. balls of the hands, palms of the hands, fingers, interdigital spaces, outer sides of the hands, fingernails) to be cleaned for a sufficient length of time by way of the mechanical rubbing together of the hands using soap or a disinfectant which is required for this. From DE 60 308 838 T2 and EP 2 015 665 B1 recording of the washing procedure by means of a camera and processor-controlled evaluation of the images in terms of whether adequate cleaning has taken place, are known. However, after evaluation, the user is only informed of whether the cleaning procedure has been carried out properly or not. If, in the opinion of the user, he/she has cleaned his/her hands sufficiently, such general feedback is not always enough to motivate the user to clean his/her hands again. Particularly in the case of users who are nursing staff, this is associated with the risk of the user himself/herself, or a person being cared for by the user, becoming infected by bacteria/viruses that may still be present on the hands.

The object of the present invention is therefore to eliminate the drawbacks set out with reference to the prior art, and, in particular, to disclose a hygiene system and a method for operating such a hygiene system, with which the hand hygiene, especially of users in the nursing sector or other critical hygiene sectors, can be improved.

This object is solved by a hygiene system and a method with the features of the respective independent claim. Advantageous further developments of the hygiene system and the method are set out in the dependent claims and in the description, wherein individual features of the advantageous further developments can be combined with each other in a technically rational manner. More particularly, the features disclosed in relation to the hygiene system can be applied to the method and vice-versa.

The object is solved, in particular, by way of a hygiene system with the features described in the introduction, in which the control is set up to determine the area of a user's hands that have been cleaned, and the control and the optical display are set up to show at least one area of the hand on the optical display if it is determined that the respective area of the hand has not, or not adequately been cleaned.

The object is also solved, in particular, by means of a method for operating a hygiene system with at least one outlet for a fluid, with a camera and with an optical display, comprising at least the following steps:
recording of the hands of a user during a cleaning procedure,
processor-supported evaluation of the recorded images, wherein it is determined which areas of the hands have been cleaned, and
showing at least one area of the hand on the optical display if it is determined that the respective area of the hand has not, or not adequately been cleaned.

Analogously, the invention therefore proposes that the user is not only shown that the hands have not been adequately cleaned, but also which areas of his/her hands have not been adequately cleaned. In this way, the user knows precisely which area of the hand has to be cleaned in an additional cleaning procedure, so that it can be assumed that users carry out an additional cleaning procedure more frequently. Consequently, hand hygiene is substantially improved. Accordingly, the optical display preferably comprises a two-dimensional screen on which an area of the hand, a hand or both hands can be shown, at least in a stylised manner, wherein through highlighting (for example outlining, colour contrasting or suchlike) it can be emphasised which specific area of the hand (for example a specific finger or specific fingers, the palm, a specific area of the palm or outer side of the hand) has not been adequately cleaned. More particularly, through displaying, it can also be emphasised whether an area of the left and/or the right hand has not been adequately cleaned.

The optical display is, in particular, arranged in such a way, that a user becomes aware of the optical display after the cleaning procedure. For example, a separate screen can be applied to a wall of the washroom or integrated into a mirror. Preferably, however, the optical display is integrated into a housing of the sanitary fitting. This facilitates the assembly of the hygiene system. Furthermore, there can be no confusion between the display and the associated washing fitting if several sanitary fittings are present in a washroom.

However, in its most general basic concept, the invention would also include the user receiving, after the washing procedure, a personalised message on a (mobile) device, on the screen of which the area of the hand that has not been cleaned is shown. For this, identification of the user as mentioned below, would also be necessary as well as a central configuration of at least part of the control as will also be described below.

The at least one camera is set up in such a way that the during a cleaning procedure for example a disinfection procedure or washing procedure), the hands of the user are recorded. In principle, for this, the camera can be arranged adjacent to the sanitary fitting. Preferably, however, the camera is integrated into a housing of the sanitary fitting, and, in particular, is directed towards the washbasin. In this way, the installation work is reduced, and on the other hand this results in a compact unit. In a preferred form of embodiment, the camera is, in particular, arranged so that a face of a user is not recorded, so that no images of the face of a user could be stored.

In particular, the sanitary fitting is a bathroom tap with at least one fluid outlet for water. A further fluid outlet for soap can be integrated into the same sanitary fitting housing. Alternatively, a soap outlet could be integrated into an adjacent housing, for example of a soap dispenser. However, the sanitary fitting can also be configured as a disinfectant dispenser for dispensing disinfectant.

In particular, the sanitary fitting comprises at least one further, second outlet for dispensing a second fluid, which differs from the first fluid, wherein both the dispensing of the first fluid from the first outlet and the dispensing of the second fluid from the second outlet can be controlled by means of the control. Dispensed in particular from the first outlet is water, as the first fluid, and dispensed in particular from the second outlet is soap, as the second fluid. In this context, the control can not only be set up to record the washing procedure and to show the result of washing, but, after detection of a user, can also control a predetermined sequence of dispensing of the fluids (in particular their order and duration).

The control can also be configured in such a way that a new cleaning procedure is initiated (for example, through the dispensing of water, soap and/or disinfectant) if it has been determined that an area of a user's hand has not been adequately cleaned. In this way, the user may possibly be motivated to clean the uncleaned area of the hand, without having to initiate the cleaning process himself/herself.

The control has an algorithm for evaluating movements of the hand and/or the distribution of the cleaning agent (for example soap or disinfectant) during the cleaning procedure. The thus determined movements of the user's hands and the distribution of the cleaning agent during the movements are compared with predetermined criteria. Alternatively or additionally, the control can also include an artificial intelligence trained to recognise an adequate cleaning procedure and trained to determine the areas of user's hand which have not been cleaned.

More particularly, it can also be evaluated whether sufficient water, soap and/or disinfectant has been used during the cleaning procedure and the result can be shown on the optical display.

If the control determines that all the areas of the user's hands have been adequately cleaned, such a perfect cleaning result can be shown on the optical display by means of suitable symbols.

In one form of embodiment, it can be envisaged that complete evaluation of the recordings takes place locally with a processor-controlled unit. In this case, the control of each sanitary fitting is thus autonomous and requires no further data connection to the internet, and in particular, to a central data processing centre (“Cloud”).

In a further form of embodiment, the local unit of the control can comprise a communication module, by means of which data can be sent to a second unit (data processing centre), more particularly connected via the internet. The second unit of the control is therefore arranged at a distance from the sanitary fitting, whereas the first unit, which is connected to the camera, is arranged locally, for example adjacent to, or in, the sanitary fitting.

When designing a communication module, it can now be envisaged that the result of an evaluation is transmitted by the first unit to the second unit so that the second unit can carry out an analysis of the transmitted data, and, in particular, an analysis of the results determined by the first unit. In this case, however, the results of the local evaluation can also be sent to the second unit in order to further improve the algorithm (reference “artificial intelligence”).

Alternatively, though, it can also be envisaged that the data recorded by the camera are effectively sent in real time by the first unit to the second unit, in which case an evaluation of the recordings takes place in the second unit, i.e. in particular in a data processing centre (reference “Cloud computing”). The result of this evaluation is then again sent to the first unit, which shows the result to a user on an optical display associated with the sanitary fitting. Such a variant has the advantage that the local unit, which is connected to the sanitary fitting, does not require great data processing power, as evaluation takes place in the second unit.

Particularly if a second unit arranged in the internet is provided, user administration can also be provided so that a user, identified before or during the cleaning procedure, receives a personalised message when the cleaning result is displayed.

However, in principle it is possible for the evaluation of the cleaning procedure to take place anonymously and the inadequately cleaned area of the hand to be shown to the (anonymous) user.

In contrast, in some cases of use, it could be desirable if the cleaning result is assigned to a specific user. Additionally, in some cases of use, it is advantageous if the user is informed of the cleaning result in a personalised, but anonymised manner. In both cases it is therefore necessary for the user to be identified (anonymously or not anonymously). In this case, the hygiene system, and, in particular, the sanitary fitting, comprises an identification device for identification of the user. The identification device can, in particular, comprise a fingerprint sensor or another sensor for recognising a physical feature of the user. Alternatively or additionally, a unit for detecting a prefabricated identification, such as a barcode, RFID chip or smartphone, can be provided. If a second unit is provided, the hygiene system can comprise a plurality of sanitary fittings, possibly installed at different locations. As a result of the connection to a central second unit, a sanitary fitting-related, a washroom-related and/or a global analysis of the evaluated data can be carried out. From such an analysis, conclusions can be drawn for improved hand hygiene measures.

Thus, particularly by way of the second unit, it can be recorded when, and for how long (on average), the hands are cleaned, how often, in the case of several cleaning procedures (especially with different cleaning agents), all areas of the hands have been cleaned, particularly in the case of consecutive cleaning procedures. It can also be determined whether there is a different success rate when using different cleaning agents, or how often it is necessary to carry out several consecutive cleaning procedures to clean the hands completely. In addition, the number of cleaning cycles a user or all users perform(s) on average until all the areas of the hands have been cleaned, can also be analysed.

It is also possible to analyse how many cleaning procedures are carried out by the sanitary fitting overall.

If a sensor for recording persons entering and leaving the washroom is also provided, the number of persons present in the washroom during a predetermined period (daily, weekly) can also be recorded. In this case it can be analysed whether every person in the washroom has carried out a cleaning procedure, or how high the proportion is.

The possibly anonymously collected data for a washroom, a building, a company or globally, can thus be made available for simple evaluation. But it is also possible to compile statistics for precisely one user, and for these to be provided to this user for his/her information. However, in areas where hygiene is critical (for example in hospitals), the cleaning procedure can be recorded in a personalised manner for all users, who have consented to being recorded, and evaluated in order to improve hand hygiene.

The invention and the technical domain are explained as examples below by way of the figures. Schematically

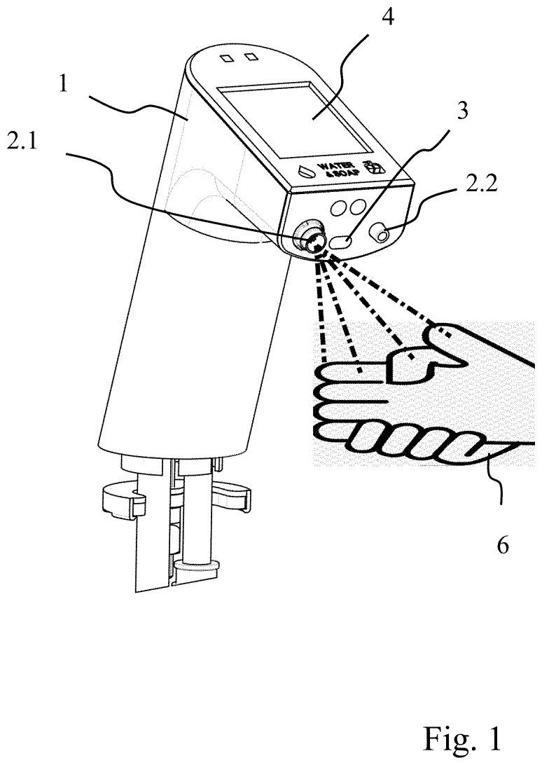

FIG. 1: shows a hygiene system with a sanitary fitting,

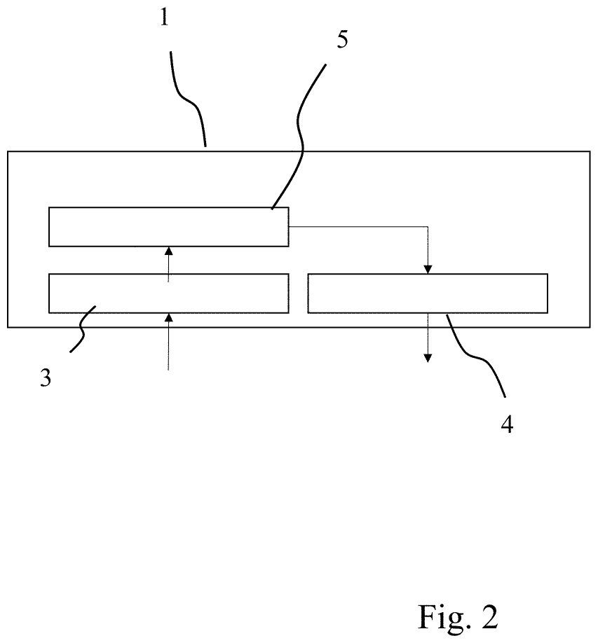

FIG. 2: shows a connection diagram of the hygiene system,

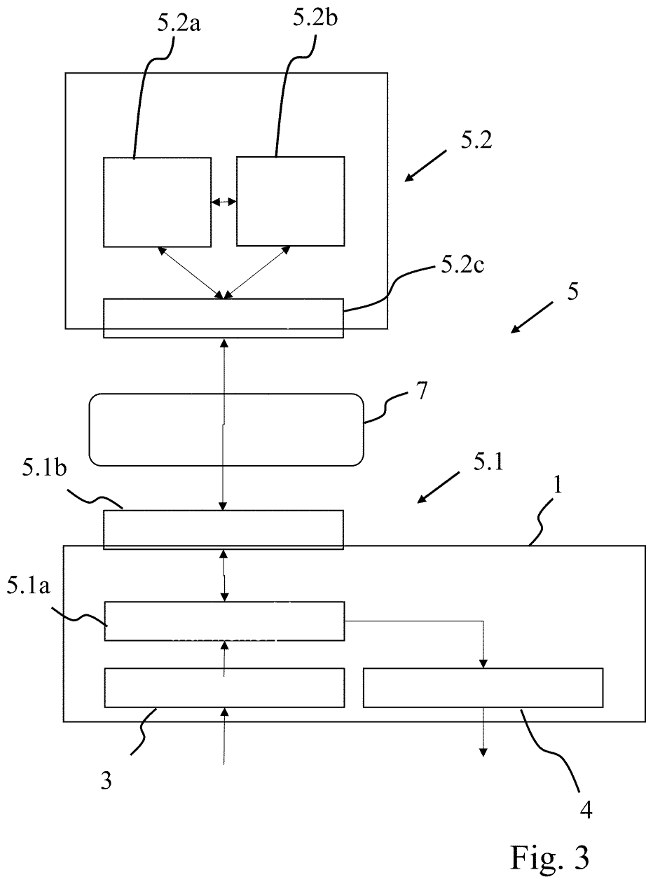

FIG. 3: shows a second form of embodiment of a hygiene system,

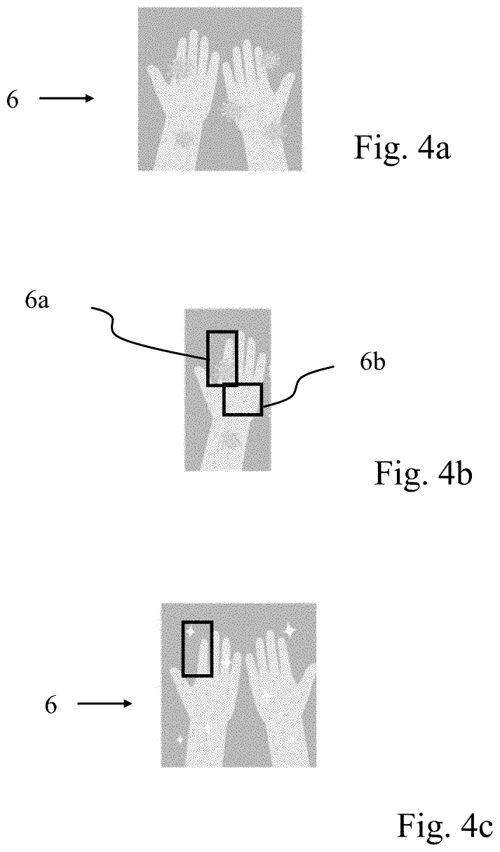

FIGS. 4a to 4c: show images of a display of the hygiene system and

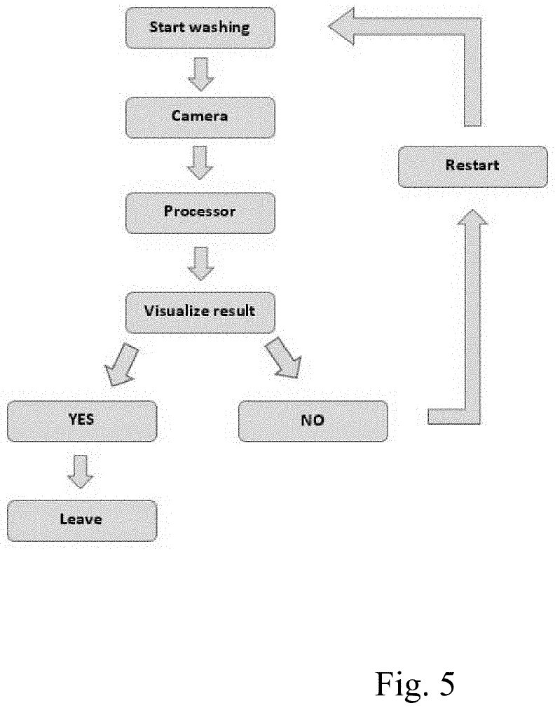

FIG. 5: shows a flow diagram of the operation of the hygiene system.

The sanitary fitting 1 shown in FIG. 1 comprises a first fluid outlet 2.1 for water and a second fluid outlet 2.2 for soap. Also integrated into the sanitary fitting 1 is a camera 3, which records the hands 6 of a user during a washing procedure. Integrated into the sanitary fitting 1 is a control, not shown in FIG. 1, which is connected to a camera 3 and an optical display 4. The optical display 4 is designed in such a way that at least stylised areas of the hand or hands can be shown thereon.

As shown in FIG. 2, the procedure of washing the hands 6 of the user is recorded, wherein the images recorded by the camera 3 are evaluated in the control 5, wherein by way of the control 5 the results are then shown on the optical display 4. In the example of embodiment shown in FIG. 2, the entire evaluation takes place in the control 5 arranged in the sanitary fitting 1.

As can be seen from the form of embodiment in FIG. 3, the control 5 can, however, also comprise several modules/units. Thus, the camera 3 integrated into the sanitary fitting 1 is connected to a data processing module 5.1a of a first unit 5.1. The first unit 5.1 associated with the sanitary fitting 1 also comprises a communication module 5.1b. The control 5 also comprises a second unit 5.2 which is formed, for example, by the data processing centre. The second unit 5.2 comprises a communication module 5.2c, a data processing unit 5.2a and a memory 5.2b. The communication modules 5.1b and 5.2c can exchange data with each other, directly or via a gateway 7.

In each case the control 5 is set up to recognise, by way of the images recorded by the camera 3, whether a user has adequately cleaned all areas 6a, 6b of the hands. If it is determined that one or more areas of the hands 6 has/have not been cleaned adequately, the area that has not been cleaned is shown on the optical display 4. The user therefore immediately knows which areas of his/her hands have to be cleaned in a further cleaning procedure.

As shown in FIG. 4a, the user can, for example, be shown that no areas of his/her hands have been adequately cleaned.

In accordance with the drawing in FIG. 4, through highlighting, the user is shown which specific areas 6a, 6b of the hands have not been adequately cleaned. If, on the other hand, it is determined that the hands 6 have been adequately cleaned, this can be indicated on the optical display 4 as shown in FIG. 4c.

FIG. 5 shows the course of such a washing procedure. After the initial start, the washing procedure is recorded with the camera 3, wherein the recorded images are evaluated in a processor-supported manner and the result of the evaluation is optically displayed in that, the inadequately cleaned area 6a, 6b of the hand is shown. It can also be envisaged that the dispensing of the cleaning agent or water is automatically restarted if an inadequately cleaned area 6a, 6b of the hand is determined. If, on the other hand, a perfect cleaning result is already achieved in the first cleaning procedure, this is also displayed and the user can leave the washroom.

In accordance with the example of embodiment shown in FIG. 2, the complete evaluation of the images recorded by the camera 3 takes place in the control 5 integrated in the sanitary fitting 1.

By contrast, in the form of embodiment shown in FIG. 3, the complete evaluation can take place in the data processing module 5.1a, and the result of the evaluation transmitted by the communication module 5.1b to the second unit 5.2. Alternatively, the images recorded by the camera 3 can be transmitted effectively in real time via the data processing module 5.1a and the communication module 5.1b to the second unit 5.2, in which evaluation of the images recorded by the camera 3 takes place. The result of the evaluation is then in turn saved in the memory 5.2b and transmitted via the communication module 5.2c and 5.1b to the first unit 5.1 so that the insufficiently cleaned areas 6a, 6b of the hands can be shown on the optical display 4.

The connection of the sanitary fitting 1 to a second unit 5.2 which is in the internee, has, in particular, the advantage that, as set out in the general description, the evaluated results can be analysed, and used in order to improve the operation of the washrooms.

### REFERENCE NUMBERS

1 Sanitary fitting

2.1, 2.2 Fluid outlet

3 Camera

4 Optical display

5 Control

5.1 First unit

5.1a Data processing module

5.1b Communication module

5.2 Second unit

5.2a Data processing unit

5.2b Memory

5.2c Communication module

6 Hands

6a, 6b Area of the hand

7 Gateway

1. Hygiene system comprising
at least one sanitary fitting (1), wherein the sanitary fitting has at least one fluid outlet (2.1, 2.2) for dispensing at least one fluid,
at least one camera (3) for recording the hands (6) of a user,
at least one optical display (4) and
a control (5), wherein the control (5) is connected to the camera (3) and the optical display (4),
characterised in that
the control (5) is set up to determine the hand areas (6a, 6b) of the hands (6) of a user which have been cleaned, and
the control (5) and the optical display (4) are set up to show at least one area of the hand (6a) on the optical display (4) if it is determined that the respective area (6a) of the hand has not, or not adequately been cleaned.

2. Hygiene system according to claim 1, wherein the control (5) is set up to show a perfect cleaning result on the optical display (4) if all areas (6a, 6b) of the hand have been adequately cleaned.

3. Hygiene system according to claim 1, wherein the sanitary fitting (1) comprises an identification device for identifying the user.

4. Hygiene system according to claim 3, wherein the identification device comprises one of the following elements:
fingerprint sensor or sensor for recognising a user by a physical feature,
unit for detecting a prefabricated identification, such as a barcode, RFID chip or smartphone,
wherein, in particular, personalised feedback to the user takes place.

5. Hygiene system according to claim 1, wherein the sanitary fitting (1) comprises at least one further, second outlet (2.2) for dispensing a second fluid, and the dispensing of the first fluid from the first outlet (2.1) and the dispensing of the second fluid from the second outlet (2.2) are controlled by means of the control (5).

6. Hygiene system according to claim 1, wherein the control (5) comprises a first unit (5.1) connected to the camera (3), and a second unit (5.2) arranged at a distance from the sanitary fitting (1).

7. Hygiene system according to claim 6, wherein the first unit (5.1) comprises a data processing module (5.1a) connected to the camera (3), and a communication module (5.1b) for communicating with the second unit (5.2).

8. Hygiene system according to claim 6, characterised in that in analyses of the second unit (5.2) at least one of the following evaluations is carried out in a data processing unit (5.2a) and/or stored in a memory (5.2b):
when and for how long the hands (6) have been cleaned,
how often in several cleaning procedures all areas (6a, 6b) of the hand have been cleaned,
number of cleaning cycles by a user until all areas (6a, 6b) of the hand have been cleaned,
number of performed cleaning procedures,
number of people in the washroom,
whether the cleaning procedure was carried out by every person in the washroom.

9. Hygiene system according to claim 1, wherein the camera (3), the optical display (4) and at least one first unit (5.1) of the control (5) are arranged in a housing of the sanitary fitting (1).

10. Method for operating a hygiene system with at least one outlet (2.1,2.2) for a fluid, with a camera (3) and with an optical display (4), more particularly for operating a hygiene system according to any one of the preceding claims, comprising at least the following steps:
recording of the hands (6) of a user during a cleaning procedure,
processor-controlled evaluation of the recording, wherein it is determined which areas (6a, 6b) of the hands (6) have been cleaned,
showing at least one area (6a) of the hand on the optical display (4) if it is determined that the respective area (6a) of the hand has not, or not adequately been cleaned.

11. Method according to claim 10, wherein a perfect cleaning result is shown on the optical display (4) if all areas of the hand (6a, 6b) have been adequately cleaned.

12. Method according to claim 10, wherein, before cleaning, the identity of the user is recorded.

13. Method according to claim 12 wherein after the cleaning procedure the user receives personalised feedback.

14. Method according to claim 10, wherein
initial processing of the recordings takes place on a data processing module (5.1a) of a first unit (5.1) which is connected to the camera,
the processed data are transmitted to a second unit (5.2),
the second unit (5.2) performs an evaluation of the processed data and
a result of the evaluation is transmitted to the first unit (5.1).

15. Method according to claim 10, characterised in that at least one of the following analyses is carried and also saved:
when and for how long the hands (6) have been cleaned,
how often in several cleaning procedures all areas (6a, 6b) of the hand have been cleaned,
number of cleaning cycles by a user until all areas (6a, 6b) of the hand are cleaned,
number of performed cleaning procedures,
number of people in the washroom,
whether the cleaning procedure was carried out by every person in the washroom.
